(19) United States
(12) Patent Application Publication
(10) Pub. No.: US 2017/0265808 A1
(43) Pub. Date: Sep. 21, 2017

(54) ORTHOPEDIC DEVICE MONITORING NODE AND SYSTEM

(71) Applicant: Alexander Frederick Wulff, Skaneateles, NY (US)

(72) Inventor: Alexander Frederick Wulff, Skaneateles, NY (US)

(21) Appl. No.: 15/416,135

(22) Filed: Jan. 26, 2017

Related U.S. Application Data
(60) Provisional application No. 62/309,894, filed on Mar. 17, 2016.

(51) Int. Cl. A61B 5/00; A61F 5/04

(52) U.S. Cl. CPC A61B 5/6812; A61F 5/04; A61B 5/0002; A61B 5/742; A61B 5/746; A61B 2562/0247; A61B 2560/0242

(57) ABSTRACT

A system and method for sensing environmental conditions within a conventional orthopedic device, such as a cast or splint, which may lead to serious medical complications from that form of treatment and to provide a notification of adverse conditions. An implantable node includes sensors for pressure, moisture and temperature, which, when combined with a power source, monitoring circuitry, communication hardware, and companion software can provide sensor data to a remote application and thus alert health care professionals to potentially dangerous environmental conditions developing within the cast.

### CROSS-REFERENCE TO RELATED APPLICATION

The present application claims priority to U.S. Provisional No. 62/309,894, filed on Mar. 17, 2016.

### BACKGROUND OF THE INVENTION

1. Field of the Invention

The present invention relates to orthopedic devices and, more specifically, to a system and method for monitoring conditions within an orthopedic cast or splint.

2. Description of the Related Art

Plaster and fiberglass casts and splints are commonly used by orthopedic specialists to treat a variety of traumatic injuries, with bone fractures being the most common indication. Casting allows both anatomical alignment of the fractured bone fragments and stability. Both conditions are critical for successful healing and restoration of function. While other treatment options exist, only casting is non-invasive, inexpensive and available to use without advanced medical facilities present. Therefore this treatment has evolved over time to be the gold standard for fracture care.

Due to the prolonged length of time casts are required, typically eight to twelve weeks, use of casts and splints can be associated with iatrogenic injury to the affected limb and patient. Feared complications include compartment syndrome, infections and skin ulceration. These complications are usually associated with detectable environmental conditions within the cast.

For example, in the case of external compartment syndrome, excess soft tissue swelling within the rigid cast can impair proper blood circulation and lead to ischemic death of tissues. Frequent measuring the skin pressure can prevent this form of compartment syndrome. Internal compartment syndrome caused by tissue swelling within the limb's fascial compartments would require the use of invasive pressure sensors inserted directly into muscle.

Infections are often heralded by fever. Temperature within the cast can be monitored and analyzed to predict possible infection. Skin ulceration or bleeding are associated with increased moisture within the cast, another detectable and correctable environmental condition.

For the alert patient who has the ability to communicate with their health care provider these complications rarely present a clinical problem. However, for the patient who is for some reason rendered unable to communicate, these complications are more frequent and often devastating. A common clinical situation is the poly-trauma with multiple injuries. They can be comatose for prolonged periods of time or sedated and intubated in an ICU setting. They often have more serious injuries than their fracture and “checking the cast” can be overlooked. In addition to comatose patients, small children often cannot communicate problems inside their cast. A few conventional systems have incorporated pressure sensors into orthopedic casts. However, none of these systems have combined the pressure sensors with other sensors and monitoring software to provide a larger spectrum of sensing capabilities, such as temperature and moisture.

Thus, an intelligent system with the ability to detect and notify the wearer, as well as physician and other medical professionals, of the onset of adverse conditions and the ability to predict harmful changes based on historical data would mitigate many of the complications that arise from wearing orthopedic casts and splints.

### BRIEF SUMMARY OF THE INVENTION

The present invention is a system and method designed to sense environmental conditions within a conventional orthopedic device, such as a cast or splint, which may lead to serious medical complications from that form of treatment and to provide a notification of adverse conditions. The device of the present invention houses sensors for pressure, moisture and temperature, which, when combined with a power source, monitoring circuitry, communication hardware, and companion software can alert health care professionals to potentially dangerous environmental conditions developing within the cast. The alert provided by the sensor may be wirelessly communicated to a health care professional via a companion mobile application preloaded and running on a portable computing device or smartphone.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of a system for monitoring orthopedic device conditions according to the present invention;

FIG. 2 is a flowchart the interconnection between a monitoring device and associated monitoring software according to the present invention;

FIG. 3 is an example of a display screen for a mobile computing device programmed according to the present invention; and

FIG. 4 is a flowchart of a method for monitoring orthopedic device conditions according to the present invention.

### DETAILED DESCRIPTION OF THE INVENTION

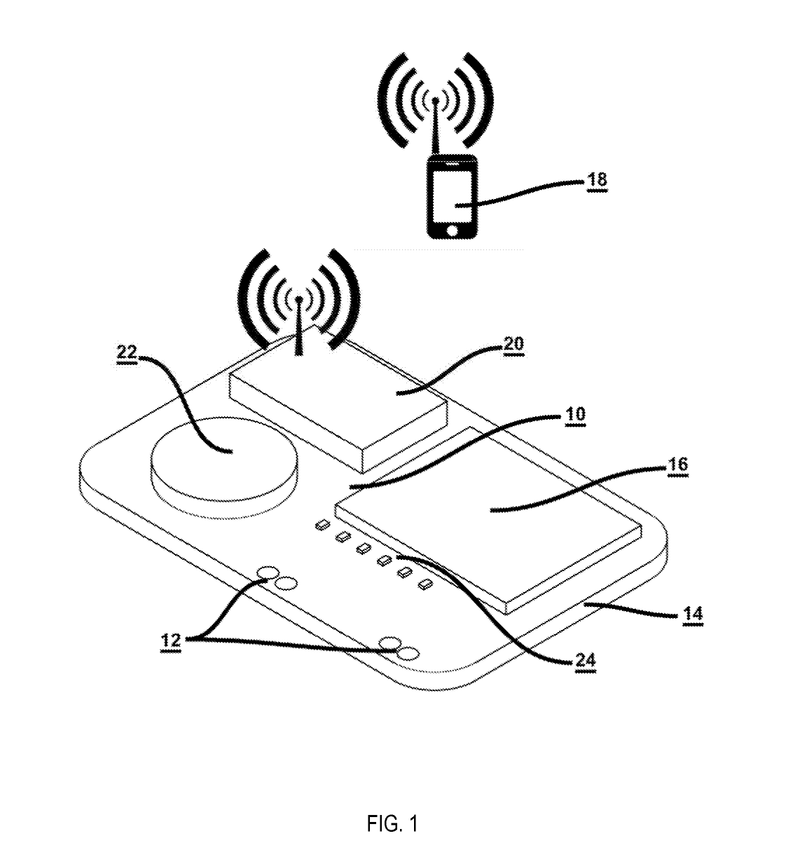

Referring to the figures, wherein like numerals refer to like parts throughout, there is seen in FIG. 1 an orthopedic monitoring node 10 for collecting data from within an orthopedic device, such as a cast or splint. Node 10 includes one or more sensors 12 mounted to a printer circuit board 14. Sensors 12 may include a force-sensing resistor or other pressure monitoring device, a resistance-based moisture sensor, a temperature sensor, and the like. A microprocessor 16, such as an ATmega or ATtiny device, is used to operate and collect data from sensors 12 and prepare collected data for transmission to a remote device 18 via a wireless communication chip 20, such as a Punch Through Design LBM313, or an NRF24L01 module. Remote device 18 may comprise a mobile phone, tablet, computer, or other receiver running an associated software application for interpreting the data and provided user notifications. A local power source 22, such as a coin cell battery or lithium ion battery, may be used to supplying power to all these components. It should be recognized that monitor may include various electronic components 24 for managing sensors 12, regulating power, etc. Multiple nodes 10 may be installed in combination with a particular orthopedic device with a single remote device 18 programmed to communicate with each node 10.

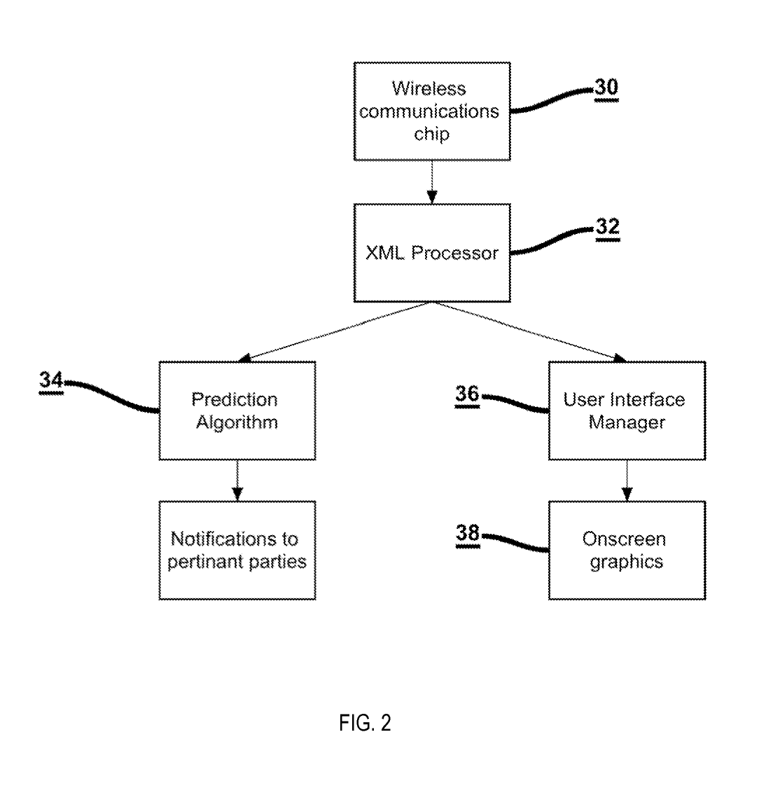

Referring to FIG. 2, remote device 18 is programmed to receive data from node 10, such as by a wireless communications chip 30 that receives an XML string object for processing by an XML processor application 32. XML processor application 32 processes the XML string to obtain all of the relevant sensor and device state data. For example, relevant data could include the readings of sensors 12, battery voltage, and other system status messages. The parsed data is passed to a prediction algorithm 34 that compares historical data and newly received data according to predetermined baselines to determine future cast conditions. For example, if the average pressure has risen in a cast by a certain number of pounds per square inch over the past few days, and this certain number is above the tolerance for such an event, algorithm 34 would mark this condition as an abnormality. As another example, if the average amount of moisture present in the cast is above the tolerance for such an event, the algorithm would mark this as another abnormality. Once algorithm 34 has determined the presence of an abnormality, or has determined that a patient is at risk of developing a future abnormality, the algorithm notifies a physician, nurse, or other medical professional via local and remote notifications. Notifications could include vibrations, messages on the screen of the receiving device, text messages, or messages on the device of a medical professional.

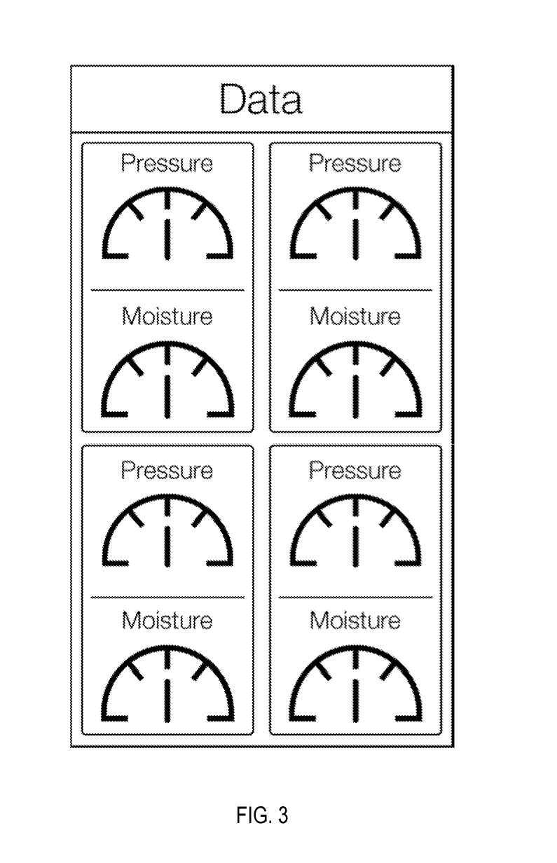

While algorithm 34 is processing new and historical data, XML processor application 32 may also pass the data to a user interface manager 36 that cooperates with onscreen graphics engine 38 to display the current status of conditions within the orthopedic device. For example, as seen in FIG. 3, onscreen graphics engine 38 may provide the sensed data as a series of gauges to provide a detailed, visual representation of current conditions inside the medical device based on all the sensor readings and state of every connected sensor node 10. Another part of the display may be devoted to displaying future projected conditions inside the cast, as predicted by algorithm 34 and discussed above. For hospital and medical offices, nodes 10 from multiple different patients may be combined into a single monitoring screen at, for example, a nursing station.

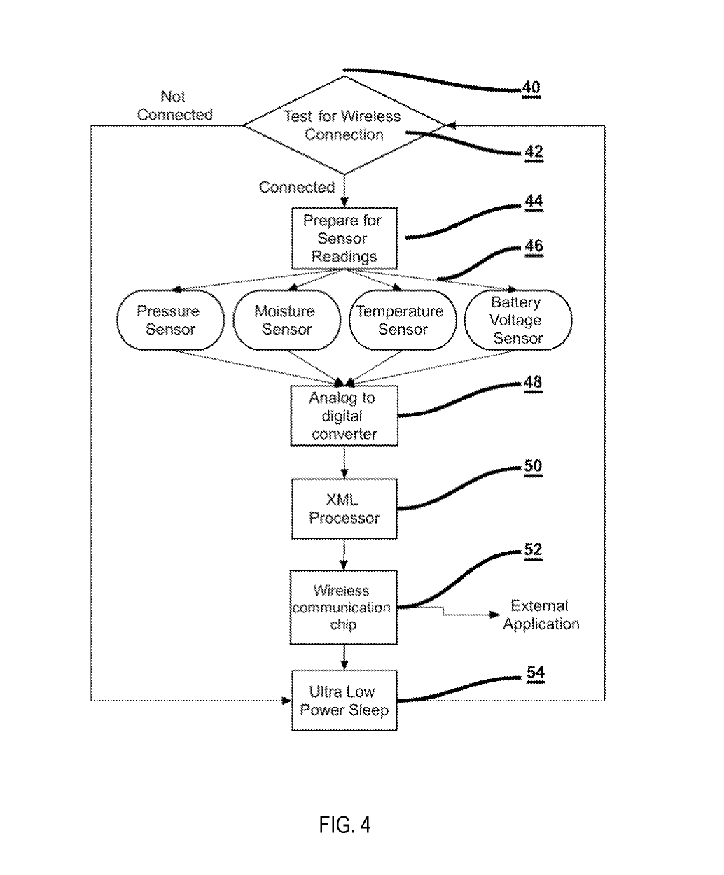

Referring to FIG. 4, node 10 may be programmed to implement a monitoring process 40 that begins with a test for an active wireless connection 42. If there is a connection, node 10 triggers sensors 12 to collect information from within orthopedic device 44. Sensors 12 then collect the associated data 46 and output signals to an analog to digital converter 48 that converts the sensor signals into digital. A local XML processor packages the digital sensor data 50 and provides the packaged data to a wireless communication chip for transmittal to the remote device 52. To conserve power, process 40 may conclude by moving into an ultra-low power sleep mode 54.

Node 10 may be programmed to execute the following high level functions:
Global Variables:
   Boolean: ConnectionState
   Integer: NodeNumber
   Integer: PressureValue
   Integer: MoistureValue
   Integer: TemperatureValue
   Integer: BatteryValue
Setup Function:
   Set sensor node name to “CastMinder”+Node Number
Looping Function:
   Get connection state of external application
   Set boolean ConnectionState to connection state of external application
   If connected to external application:
   Read sensor value for pressure sensor
   Set integer PressureValue to read value of pressure sensor
   Read sensor value for moisture sensor
   Set integer MoistureValue to read value of moisture sensor
   Read sensor value for temperature sensor
   Set integer TemperatureValue to read value of temperature sensor
   Read sensor value for battery voltage sensor
   Set integer BatteryValue to read value of battery sensor
   Create String DataString to send to external application
   Set DataString equal to the following, substituting variable names as necessary:

“<CastMinder Data>

<pressure>BatteryValue</pressure>
<moisture>MoistureValue</moisture>
<temperature>TemperatureValue</temperature>
<battery>BatteryValue</battery>
<nodeID>NodeNumber</nodeID>

</CastMinder Data>”

Encode DataString into binary using UTF-8 encoding
Send binary over Bluetooth to connected external application
Sleep for one second
Loop program back to beginning of “Looping Function”

Remote device 18 may be programmed to execute the following high level functions:
Received Data:
   Convert binary data into string with UTF-8 encoding
   Separate XML string into component XML data parts
   If data contains pressure, moisture, temperature, and battery values:
      Set Integer DateReceived to current date value
      Set String SensorName to the name of the sending sensor node
      Append data with DateReceived and SensorName
      Add new sensor data to SQL database
      Post New Data Notification to rest of application
New Data Notification Received—User Interface Manager:
   Parse SQL database for most recent sensor data reading
   Split sensor reading into component sensor values
   Display each sensor reading on an on-screen gauge

As described above, the present invention may be a system, a method, and/or a computer program associated therewith and is described herein with reference to flowcharts and block diagrams of methods and systems. The flowchart and block diagrams illustrate the architecture, functionality, and operation of possible implementations of systems, methods, and computer programs of the present invention. It should be understood that each block of the flowcharts and block diagrams can be implemented by computer readable program instructions in software, firmware, or dedicated analog or digital circuits. These computer readable program instructions may be implemented on the processor of a general purpose computer, a special purpose computer, or other programmable data processing apparatus to produce a machine that implements a part or all of any of the blocks in the flowcharts and block diagrams. Each block in the flowchart or block diagrams may represent a module, segment, or portion of instructions, which comprises one or more executable instructions for implementing the specified logical functions. It should also be noted that each block of the block diagrams and flowchart illustrations, or combinations of blocks in the block diagrams and flowcharts, can be implemented by special purpose hardware-based systems that perform the specified functions or acts or carry out combinations of special purpose hardware and computer instructions.

What is claimed is:

1. An orthopedic device monitoring system, comprising:
at least one sensor mounted to a substrate for producing a signal reflecting a condition within a cast that is to be monitored by the sensor;
a wireless communication module mounted to the substrate;
a microprocessor mounted to the substrate and interconnected to the sensor and the wireless communication module, wherein the microprocessor is programmed to sample the signal reflecting the at least one condition monitored by the sensor and to wirelessly communicate data representing the at least one condition through the wireless communication module.

2. The system of claim 1, wherein the sensor is selected from the group consisting of a pressure sensor, a temperature sensor, a humidity sensor, and combinations thereof.

3. The system of claim 2, further comprising a remote device wirelessly connected to the sensor.

4. The system of claim 3, wherein the remote device includes a second microprocessor programmed to receive the data representing the at least one condition from the at least one sensor.

5. The system of claim 4, wherein the second microprocessor of the remote device is programmed to display the data representing the at least one condition to a user of the remote device after the data is received from the at least one sensor.

6. The system of claim 5, wherein the second microprocessor of the remote device is programmed to track the data representing the at least one condition after the data is received from the at least one sensor.

7. The system of claim 6, wherein the second microprocessor of the remote device is programmed to identify if the data representing the at least one condition indicates an abnormality inside the cast.

8. The system of claim 6, wherein the second microprocessor of the remote device is programmed to provide a notification if the data representing the at least one condition indicates an abnormality inside the cast.

9. A method of monitoring conditions inside an orthopedic device, comprising the steps of:
providing a sensor node including at least one sensor mounted to a substrate for producing a signal reflecting a condition within a cast that is to be monitored by the sensor, a wireless communication module mounted to the substrate, and a microprocessor mounted to the substrate and interconnected to the sensor and the wireless communication module; and
wirelessly transmitting the data representing the at least one condition through the wireless communication module to a remote device.

10. The method of claim 9, wherein the sensor is selected from the group consisting of a pressure sensor, a temperature sensor, a moisture sensor, and combinations thereof.

11. The method of claim 10, further comprising the step of connecting a remote device wirelessly to the sensor.

12. The method of claim 11, further comprising the step of receiving with the remote device the data representing the at least one condition from the at least one sensor.

13. The method of claim 12, further comprising the step of using the remote device to display the data representing the at least one condition to a user of the remote device after the data is received from the at least one sensor.

14. The method of claim 13, further comprising the step of tracking the data representing the at least one condition after the data is received from the at least one sensor.

15. The method of claim 14, further comprising the step of identifying if the data representing the at least one condition indicates an abnormality inside the cast.

16. The method of claim 15, further comprising the step of providing a notification if the data representing the at least one condition indicates an abnormality inside the cast.
